SEQUENCE LISTING (20 sequences)

SEQ ID NO: 1 - protein, 697 aa, Homo sapiens
  Human HGF amino acid sequence having no secretion signal sequence
QRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 2 - protein, 179 aa, Homo sapiens
  Human NK1 amino acid sequence having no secretion signal sequence
QRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVE

SEQ ID NO: 3 - protein, 31 aa, Homo sapiens
  Human HGF-derived secretion signal sequence
MWVTKLLPALLLQHVLLHLLLLPIAIPYAEG

SEQ ID NO: 4 - protein, 7 aa, Artificial
  Amino acid sequence of cysteine residue and 6xHis tag, added to the amino terminus of human NK1 protein
CHHHHHH

SEQ ID NO: 5 - protein, 7 aa, Artificial
  Amino acid sequence of 6xHis tag and cysteine residue, added to the carboxyl terminus of human NK1 protein
HHHHHHC

SEQ ID NO: 6 - DNA, 2576 bp, Homo sapiens
  CDS (102)..(2288)
gggctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataa

SEQ ID NO: 7 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 8 - protein, 12 aa, Homo sapiens
SPLAQAVRSSSR

SEQ ID NO: 9 - protein, 9 aa, Artificial
  Artificial ADAM-17 cleavable  sequence
PRAAAVKSP

SEQ ID NO: 10 - protein, 9 aa, Homo sapiens
QETNRSFSK

SEQ ID NO: 11 - protein, 9 aa, Homo sapiens
PRVAQVSIT

SEQ ID NO: 12 - protein, 9 aa, Homo sapiens
PVAAAVVSH

SEQ ID NO: 13 - protein, 9 aa, Homo sapiens
ADLLAVVAA

SEQ ID NO: 14 - protein, 9 aa, Artificial
  Variant sequence 1 of SEQ No.9
PRAAALKSP

SEQ ID NO: 15 - protein, 9 aa, Artificial
  Variant sequence 2 of SEQ No.9
PRAAAYKSP

SEQ ID NO: 16 - protein, 9 aa, Artificial
  Variant sequence 3 of SEQ No.9
PRAAAAKSP

SEQ ID NO: 17 - protein, 11 aa, Artificial
  Artificial Thrombin cleavable  sequence
TFLTPRGVRLG

SEQ ID NO: 18 - protein, 9 aa, Artificial
  Artificial Thrombin cleavable  sequence
TFPPRSFLG

SEQ ID NO: 19 - protein, 8 aa, Homo sapiens
  Thrombin cleavable sequence derived from Human Prothrombin
FNPRTFGS

SEQ ID NO: 20 - protein, 8 aa, Homo sapiens
  Thrombin cleavable sequence derived from Human HGFA
LRPRIIGG

(19) United States
(12) Patent Application Publication
(10) Pub. No.: US 2022/0220176 A1
(43) Pub. Date: Jul. 14, 2022

(54) POLYETHYLENE GLYCOL-MODIFIED FORM OF HEPATOCYTE GROWTH FACTOR OR ACTIVE FRAGMENT THEREOF

(71) Applicant: Toray Industries, Inc., Tokyo (JP)

(72) Inventors: Takashi Serizawa, Kamakura-shi, Kanagawa (JP); Katsuyuki Mori, Kamakura-shi, Kanagawa (JP); Tomomi Asano, Kamakura-shi, Kanagawa (JP); Mikiya Sato, Kamakura-shi, Kanagawa (JP)

(21) Appl. No.: 17/421,417

(22) PCT Filed: Jan. 28, 2020
(86) PCT No.: PCT/JP2020/002864

§ 371 Date: Jul. 8, 2021

(30) Foreign Application Priority Data
Jan. 28, 2019 (JP) 2019-011808

(51) Int. Cl. C07K 14/475; A61K 47/60

(52) U.S. Cl. CPC C07K 14/4753; A61K 38/00; A61K 47/60

(57) ABSTRACT

A variant of a hepatocyte growth factor or an active fragment thereof reduces the liver tropism and selectively exert bioactivity in a target tissue in disease other than a liver tissue. A polyethylene glycol-modified form of a hepatocyte growth factor or an active fragment thereof has polyethylene glycol(s) bound to (a) terminus(es) of the hepatocyte growth factor or the active fragment thereof via (a) protease-sensitive peptide(s).

### TECHNICAL FIELD

This disclosure relates to a polyethylene glycol-modified form of a hepatocyte growth factor or an active fragment thereof.

### BACKGROUND

Hepatocyte growth factor is a growth factor having diverse pharmacological effects and are known to have an anti-apoptotic effect, an angiogenic effect, a vasodilatory effect, an anti-organ fibrosis effect, an anti-epithelial mesenchymal transition effect, and the like, in addition to an originally found hepatocyte proliferative effect. Currently, its clinical applications to various diseases have been attempted.

However, it has been suggested that the hepatocyte growth factor needs to be frequently administered in large amounts to sustain their pharmacological effects because the hepatocyte growth factor has an in vivo half-life as short as approximately 30 minutes (Liu K. X. et al., The American Journal of Physiology, 1998, Vol. 275, pp E835-E842). Meanwhile, it has been reported that administration of the hepatocyte growth factor in large amounts induces an enlargement of the liver ascribable to the hepatocyte proliferation as an adverse effect thereof, because the hepatocyte growth factor has a liver tropism (Sakata H. et al., Cell Growth & Differentiation, 1996, Vol. 7, pp 1513-1523).

NK1 composed of N domain and kringle 1 in Jakubczak J. L. et al., Molecular and Cellular Biology, 1998, Vol. 18, No. 3, pp 1275-1283, and NK2 composed of N domain, kringle 1 and kringle 2 in Otsuka T. et al., Molecular and Cellular Biology, 2000, Vol. 20, No. 6, pp 2055-2065 have been reported as active fragments that are natural splicing variants of the hepatocyte growth factor. Also, NK4 composed of N domain, kringle 1, kringle 2, kringle 3 and kringle 4 has been reported as an active fragment developed by a recombination technique in Date K. et al., FEBS Letters, 1997, Vol. 420, No. 1, pp 1-6. It has been reported that those active fragments have an agonistic activity or an antagonistic activity against c-Met, a receptor tyrosine kinase for hepatocyte growth factor receptor, in vitro and in vivo. It has also been reported that NK1 has a liver tropism and induces an enlargement of the liver in vivo (Jakubczak J. L. et al., Molecular and Cellular Biology, 1998, Vol. 18, No. 3, pp 1275-1283 and Ross J. et al., Gastroenterology, 2012, Vol. 142, pp 897-906).

Polyethylene glycol-modified forms aimed mainly at an in vivo half-life extending effect have been reported as to the hepatocyte growth factors and NK4, one of the active fragments thereof (International Publication No. WO 1996/28475 and JP Patent Publication (Kokai) No. 2010-174034 A (2010)). Polyethylene glycol is a highly biocompatible polymer and is widely used as a modifying agent aimed at in vivo half-life extension or immunogenicity reduction of proteins.

In chemically modifying a protein with the polyethylene glycol, it is known that the bioactivity of the protein is decreased or lost, depending on the position of the modification. WO 1996/28475 has reported that, for example, a modified form in which a hepatocyte growth factor is chemically modified directly with a plurality of polyethylene glycol molecules at random can achieve an in vivo half-life extending effect on the hepatocyte growth factor, whereas its bioactivity is lowered by 30% or more.

Stefan N. et al., Bioconjugate Chemistry, 2014, Vol. 25, pp 2144-2156 has reported a prodrug of Pseudomonas aeruginosa-derived exotoxin A chemically modified with polyethylene glycol via a protease-sensitive peptide for rhinovirus-derived 3C protease for the purpose of empirically controlling drug-derived toxicity.

In Stefan N. et al., Bioconjugate Chemistry, 2014, Vol. 25, pp 2144-2156, however, in vivo and ex vivo functional verification has not been carried out on a prodrug of exotoxin A which is a toxin protein derived from Pseudomonas aeruginosa. Furthermore, the prodrug of exotoxin A disclosed in Stefan N. et al., Bioconjugate Chemistry, 2014, Vol. 25, pp 2144-2156 has undergone chemical modification with polyethylene glycol via a protease-sensitive peptide at two locations, i.e., at an end of exotoxin A and in the middle of its sequence, to suppress the bioactivity of the exotoxin A. In general, peptide addition or chemical modification with polyethylene glycol in the middle of the sequence of a protein entails uncertainty from the viewpoint of the maintenance of bioactivity originally possessed by the protein because of a high level of difficulty in production and because an unnecessary amino acid sequence derived from the protease-sensitive peptide remains in the middle of the structure of the protein even after cleavage by the protease. Thus, the technique described in Stefan N. et al., Bioconjugate Chemistry, 2014, Vol. 25, pp 2144-2156 has poor generality and seems to be not easily applicable to other proteins.

Stefan N. et al., Bioconjugate Chemistry, 2014, Vol. 25, pp 2144-2156 neither discloses nor suggests a prodrug of a hepatocyte growth factor or an active fragment thereof and neither discloses nor suggests in vivo protease that may be exploited for the design of the prodrug for the purpose of reducing liver tropism of the hepatocyte growth factor or the active fragment thereof.

Hence, it has been desired to develop a variant of a hepatocyte growth factor or an active fragment thereof which can reduce liver tropism of the hepatocyte growth factor or the active fragment thereof and selectively exert the bioactivity of the hepatocyte growth factor or the active fragment thereof in a target disease tissue other than a liver tissue.

Accordingly, it could be helpful to provide a variant of a hepatocyte growth factor or an active fragment thereof which can address the issues described above.

### SUMMARY

We generated a hepatocyte growth factor or an active fragment thereof, which is chemically modified with the polyethylene glycol(s) via (a) protease-sensitive peptide(s), whereby liver tropism of the hepatocyte growth factor or the active fragment thereof can be reduced while the bioactivity of the hepatocyte growth factor or the active fragment thereof can be selectively exerted by the cleavage of the protease-sensitive peptide in a target disease tissue.

We thus provide (1) to (7):

(1) A polyethylene glycol-modified form of a hepatocyte growth factor or an active fragment thereof, wherein the polyethylene glycol(s) is/are bound to any end of the hepatocyte growth factor or the active fragment thereof via (a) protease-sensitive peptide(s).

(2) The polyethylene glycol-modified form of a hepatocyte growth factor or an active fragment thereof according to (1), wherein the protease-sensitive peptide(s) is/are (an) ADAM17-sensitive peptide(s) or (a) thrombin-sensitive peptide(s).

(3) The polyethylene glycol-modified form of a hepatocyte growth factor or an active fragment thereof according to (1), wherein the protease-sensitive peptide(s) has/have an amino acid sequence represented by any one of SEQ ID NOs: 8 to 20 in the sequence listing.

(4) The polyethylene glycol-modified form of a hepatocyte growth factor or an active fragment thereof according to any of (1) to (3), wherein (a) number-average molecular weight(s) of the polyethylene glycol(s) is/are 20000 to 100000.

(5) The polyethylene glycol-modified form of a hepatocyte growth factor or an active fragment thereof according to any of (1) to (4), wherein the active fragment of the hepatocyte growth factor is NK1.

(6) The polyethylene glycol-modified form of a hepatocyte growth factor or an active fragment thereof according to any of (1) to (5), wherein the active fragment of the hepatocyte growth factor has the amino acid sequence represented by SEQ ID NO: 2 in the sequence listing.

(7) A medicament comprising a polyethylene glycol-modified form of a hepatocyte growth factor or an active fragment thereof according to any of (1) to (6) as an active ingredient.

Our polyethylene glycol-modified form of a hepatocyte growth factor or an active fragment thereof can reduce liver tropism originally possessed by the hepatocyte growth factor or the active fragment thereof and selectively exert the pharmacological effects of the hepatocyte growth factor or the active fragment thereof by exerting its bioactivity in a target disease tissue of the kidney or the like.

### BRIEF DESCRIPTION OF THE DRAWINGS

FIG. 1 is a diagram showing the amounts of enzymatic activity exerted by ADAM17 protein in kidney tissues and liver tissues of kidney disease model mice and normal mice.

FIG. 2 is a diagram showing the expression levels of ADAM17 protein in kidney tissues and liver tissues of kidney disease model mice and normal mice. FIG. 2(a) shows a Western blotting image of the ADAM17 protein, and FIG. 2(b) is a diagram showing a numerical values calculated from their band intensities.

FIG. 3 is a diagram showing the releases of the active form, human NK1, from the prodrugs of human NK1 by protease treatment.

FIG. 4 is a diagram showing the attenuations of the HGF activity of human NK1 by the prodrug modifications of protease-sensitive peptide-added human NK1.

FIG. 5 is a diagram showing the tissue-selective releases of the active form, human NK1, from the prodrugs of human NK1.

FIG. 6 is a diagram showing the in vivo tissue-selective bioactivity of the prodrug of human NK1.

FIG. 7 is a diagram showing the comparison of activity controls of the prodrugs of human NK1, depending on difference in polyethylene glycol modification site.

### DETAILED DESCRIPTION

Polyethylene glycol-Modified Form of Hepatocyte Growth Factor or Active Fragment Thereof

In our polyethylene glycol-modified form of a hepatocyte growth factor or an active fragment thereof (hereinafter, also the PEG-modified form of the HGF or an active fragment thereof), polyethylene glycol(s) (hereinafter, also PEG) is/are covalently bound to (a) terminus(es) (amino terminus and/or carboxyl terminus) of one molecule of the hepatocyte growth factor (hereinafter, also HGF) or the active fragment thereof via (a) protease-sensitive peptide(s).

One example of the PEG-modified form of the HGF or an active fragment thereof includes a form in which one or more molecules of PEG are covalently bound to (a) terminus(es) (amino terminus or carboxyl terminus) of the HGF or the active fragment thereof via (a) protease-sensitive peptide(s). In this respect, the binding mode between the terminus(es) of the HGF or the active fragment thereof and the protease-sensitive peptide(s) is not particularly limited. They may be bound directly or may be arbitrarily bound via (a) spacer sequence(s). The binding mode between the protease-sensitive peptide(s) and the PEG is not particularly limited. The PEG may be bound directly to the protease-sensitive peptide(s) or may be arbitrarily bound thereto via an amino acid or the like artificially added to the protease-sensitive peptide(s). Further, a protease-sensitive peptide for purification or a tag sequence for purification may be contained between the protease-sensitive peptide and the artificially added amino acid or the like. Examples thereof include a form in which a terminus of the HGF or an active fragment thereof is covalently bound to the carboxyl terminus of a protease-sensitive peptide and the PEG is covalently bound to the amino terminus of the protease-sensitive peptide, and a form in which a terminus of the HGF or an active fragment thereof is covalently bound to the amino terminus of a protease-sensitive peptide and the PEG is covalently bound to the carboxyl terminus of the protease-sensitive peptide. A form is preferred in which a terminus of the HGF or an active fragment thereof is covalently bound to the carboxyl terminus of a protease-sensitive peptide and the PEG is covalently bound to the amino terminus of the protease-sensitive peptide.

The amino terminus of the HGF or the active fragment thereof is responsible for the functions of dimerization of the HGF or the active fragment thereof that are important for controlling the bioactivity of the HGF or the active fragment thereof, and is also responsible for liver tropism of the HGF or the active fragment thereof. Therefore, to reduce bioactivity and liver tropism possessed by the HGF or the active fragment thereof, a form is preferred in which one molecule of PEG is covalently bound to the amino terminus of the HGF or an active fragment thereof via a protease-sensitive peptide; a form is more preferred in which the carboxyl terminus of a protease-sensitive peptide is covalently bound to the amino terminus of the HGF or an active fragment thereof and further, one molecule of PEG is covalently bound to the amino terminus of the protease-sensitive peptide; a form is further preferred in which the carboxyl terminus of an ADAM17-sensitive peptide or a thrombin-sensitive peptide is covalently bound to the amino terminus of the HGF or an active fragment thereof and further, one molecule of PEG is covalently bound to the amino terminus of the ADAM17-sensitive peptide or the thrombin-sensitive peptide; a form is still further preferred in which the carboxyl terminus of an ADAM17-sensitive peptide or a thrombin-sensitive peptide is covalently bound to the amino terminus of NK1, an active fragment of HGF, and further, one molecule of PEG having a number-average molecular weight of 20000 to 100000 is covalently bound to the amino terminus of the ADAM17-sensitive peptide or the thrombin-sensitive peptide; and a form is most preferred in which the carboxyl terminus of an ADAM17-sensitive peptide or a thrombin-sensitive peptide is covalently bound to the amino terminus of NK1 and further, one molecule of tetrabranched PEG having a number-average molecular weight of 70000 to 90000 is covalently bound to the amino terminus of the ADAM17-sensitive peptide or the thrombin-sensitive peptide. An arbitrary spacer sequence may be added to between the HGF or the active fragment thereof and the protease-sensitive peptide to control cleavage efficiency by protease.

Another example of the PEG-modified form of the HGF or an active fragment thereof also include a form in which one molecule of PEG is covalently bound to the middle of the sequence of the HGF or the active fragment thereof via a protease-sensitive peptide. HGF and active fragment thereof

HGF is composed of N domain, kringle 1, kringle 2, kringle 3, kringle 4 and SPH domain in this order from the amino-terminal side. The N domain, kringle 1, kringle 2, kringle 3 and kringle 4 constitute an α chain, and the SPH domain constitutes a β chain. HGF is biosynthesized as single-chain pro-HGF and secreted along with the removal of its secretory signal sequence. Then, the resulting sequence is extracellularly processed, between an 494^{th} arginine residue (R494) and a 495^{th} valine residue (V495) counted from initiating methionine, by protease to form a heterodimer chain of the α chain and the β chain bound through a disulfide bond, which is active (Miyazawa K. et al., The Journal of Biological Chemistry, 1996, Vol. 271, No. 7, p. 3615-3618). The HGF means an active HGF having bioactivity.

It is known that diverse pharmacological effects of the HGF such as a hepatocyte proliferative effect, an anti-apoptotic effect, an angiogenic effect, a vasodilatory effect, an anti-organ fibrosis effect, and an anti-epithelial mesenchymal transition effect, are induced by the binding of the HGF to c-Met, a receptor tyrosine kinase for the HGF. Through this binding of HGF to c-Met, a plurality of tyrosine residues on the intracellular side of the c-Met are phosphorylated, and signaling molecules bind to the phosphorylated tyrosine residues to activate the signaling pathway. In this respect, 1234^{th} and 1235^{th} tyrosine residues (Y1234/1235) are known as the particularly important phosphorylation sites.

Human HGF is biosynthesized as a secretory protein consisting of 728 amino acid residues (containing a secretory signal sequence (31 amino acid residues from initiating methionine)) (GenBank accession No: M29145) and, when secreted, becomes a protein consisting of 697 amino acid residues (SEQ ID NO: 1) by the removal of the secretory signal sequence.

The HGF described above encompasses a HGF having the same amino acid sequence as that of naturally occurring HGF (hereinafter, also natural HGF), as well as an amino acid mutant of HGF having an amino acid sequence derived from the amino acid sequence of natural HGF by the deletion, substitution or addition (or insertion) of one or several amino acids and having bioactivity as HGF, and further encompasses even a HGF having an altered sugar chain moiety of natural HGF and a HGF having no sugar chain moiety. The amino acid mutant of HGF is preferably a mutant having 90% or higher sequence identity to the amino acid sequence of natural HGF, more preferably a mutant having 95% or higher sequence identity thereto, further preferably a mutant having 98% or higher sequence identity. Examples of the amino acid mutant of HGF include a HGF with a deletion of 5 amino acid residues within kringle 1 (which is a naturally occurring mutant; hereinafter, also referred to as deleted variant of HGF) (Kinosaki M. et al., FEBS Letters, Vol. 434, 1998, p. 165-170), which has been reported to have higher specific activity than that of human HGF (SEQ ID NO: 1) in certain cell lines.

Conservative amino acid substitution generally refers to substitution between amino acids similar in chemical properties, electric properties (or polarity and/or hydrophobicity) or structural properties. Since such substitution can suppress a marked change in conformation of a polypeptide including natural HGF, the polypeptide can retain its activity without large impairment and, in sometimes, can have higher activity than the natural one. Specific examples of such amino acid substitution include substitution between acidic amino acids (e.g., aspartic acid (D) and glutamic acid (E)), substitution between basic amino acids (e.g., histidine (H), lysine (K) and arginine (R)), substitution between aromatic amino acids (e.g., phenylalanine (F), tyrosine (Y) and tryptophan (W)), substitution between hydrophilic amino acids (e.g., cysteine (C), aspartic acid (D), glutamic acid (E), histidine (H), lysine (K), asparagine (N), glutamine (Q), arginine (R), serine (S) and threonine (T)), and substitution between hydrophobic amino acids (e.g., alanine (A), phenylalanine (F), isoleucine (I), leucine (L), norleucine (Nle), methionine (M), valine (V), tryptophan (W) and tyrosine (Y)).

The HGF described above also encompasses a recombinant HGF prepared by a gene recombination technique on the basis of the amino acid sequence or nucleotide sequence of natural HGF.

The “sequence identity” refers to identity between two sequences that can be determined using an algorithm such as BLAST or FASTA, in which the two sequences are aligned to attain the maximum degree of identity with or without gaps involved, and can generally be calculated as the percentage (%) of the number of identical amino acids to the total number of amino acids (including gaps) (Altschul S. et al., Journal of Molecular Biology, 1990, Vol. 215, No. 3, p. 403-410; and Altschul S. et al., Nucleic Acids Research, 1997, Vol. 25, No. 17, p. 3389-3402).

The term “several” refers to an integer of 2 to 10, i.e., 2, 3, 4, 5, 6, 7, 8, 9 or 10.

The active fragment of HGF refers to a protein that has a portion of the structure of HGF and exerts bioactivity (agonistic activity) as HGF by binding to c-Met, or a protein that acts as an antagonist (exerts antagonistic activity) against c-Met.

Examples of the active fragment of HGF described above include NK1 and NK2 that are natural splicing variants of HGF, and NK4 developed by a recombination technique. NK1 is composed of N domain and kringle 1 on the amino-terminal side of HGF, and NK2 is composed of N domain, kringle 1 and kringle 2 on the amino-terminal side of HGF. It has been reported that each of these fragments acts as an agonist or an antagonist against c-Met in vivo (Jakubczak J. L. et al., Molecular and Cellular Biology, Vol. 18, No. 3, 1998, p. 1275-1283; and Otsuka T. et al., Molecular and Cellular Biology, Vol. 20, No. 6, 2000, p. 2055-2065). It has also been reported that NK1 has liver tropism and induces liver hypertrophy in vivo (Jakubczak J. L. et al., Molecular and Cellular Biology, 1998, Vol. 18, No. 3, p. 1275-1283; and Ross J. et al., Gastroenterology, 2012, Vol. 142, p. 897-906). NK4 is composed of N domain, kringle 1, kringle 2, kringle 3 and kringle 4 on the amino-terminal side of HGF and has been reported to act as an antagonist against c-Met (Date K. et al., FEBS Letters, Vol. 420, No. 1, 1997, p. 1-6).

The active fragment of HGF described above encompasses an active fragment having the same amino acid sequence as that derived from natural HGF, as well as a mutant having an amino acid sequence derived from the amino acid sequence of natural HGF by the deletion, substitution or addition of one or several amino acids and having bioactivity (agonistic activity) as HGF or antagonistic activity against c-Met, and further encompasses even a mutant having an altered sugar chain moiety derived from natural HGF and a mutant having no sugar chain moiety derived from natural HGF. The mutant of the active fragment of HGF is preferably a mutant having 90% or higher sequence identity to the amino acid sequence derived from natural HGF, more preferably a mutant having 95% or higher sequence identity thereto, further preferably a mutant having 98% or higher sequence identity. Examples of the highly active NK1 mutant include 1K1 (Lietha D. et al., The EMBO Journal, 2001, Vol. 20, No. 20, p. 5543-5555) and M2.2 (Jones D. S. 2nd. et al., Proceedings of the National Academy of Sciences of the United States of America, 2011, Vol. 108, No. 32, p. 13035-13040).

The HGF or the active fragment thereof contained in the PEG-modified form of HGF or an active fragment thereof is preferably natural HGF (also including a mutant such as deleted variant of HGF), NK1 (also including a mutant), NK2 (also including a mutant) or NK4 (also including a mutant), more preferably NK1 (also including a mutant) or NK2 (also including a mutant), further preferably NK1 (also including a mutant), most preferably human NK1 consisting of the amino acid sequence represented by SEQ ID NO: 2. The amino acid sequence represented by SEQ ID NO: 2 excludes a secretory signal sequence (MWVTKLLPALLLQHVLLHLLLLPIAIPYAEG: SEQ ID NO: 3) derived from human HGF.

The HGF or the active fragment thereof described above contains a mammal-derived amino acid sequence, which is preferably a human-, cat- or dog-derived amino acid sequence, more preferably a human-derived amino acid sequence.

The HGF or the active fragment thereof can be obtained, for example, according to a gene recombination technique known in the art by designing a nucleic acid sequence (DNA) encoding the HGF or the active fragment thereof, and transiently or stably introducing the nucleic acid sequence to cells for expression.

One to 20 amino acids may be added as a spacer sequence to between the HGF or the active fragment thereof and the protease-sensitive peptide by use of a gene recombination technique known in the art. The HGF or the active fragment thereof with the spacer sequence between the HGF or the active fragment thereof and the protease-sensitive peptide can be obtained, for example, according to a gene recombination technique known in the art by adding a nucleic acid (DNA) sequence encoding an arbitrarily selected spacer sequence to a nucleic acid (DNA) sequence encoding the HGF or the active fragment thereof to be consecutive, further adding a nucleic acid (DNA) sequence encoding the protease-sensitive peptide thereto to be consecutive, and transiently or stably introducing the resulting sequence to cells for expression. Examples of the amino acid that can be used in the spacer sequence include aspartic acid (D), glutamic acid (E), histidine (H), lysine (K), arginine (R), phenylalanine (F), tyrosine (Y), tryptophan (W), alanine (A), cysteine (C), asparagine (N), glutamine (Q), serine (S), threonine (T), isoleucine (I), leucine (L), norleucine (Nle), methionine (M), valine (V) and tryptophan (W). The spacer sequence used is appropriately selected in consideration of the cleavage efficiency of the protease-sensitive peptide, or the activity control efficiency of the HGF or the active fragment thereof by PEG modification.

### Protease-Sensitive Peptide

The protease-sensitive peptide means a peptide cleaved by a protease whose expression is found in a target disease tissue other than a liver tissue and is relatively low in the liver tissue.

The protease corresponding to the protease-sensitive peptide needs to be selected as an appropriate one by evaluating a relative difference in enzymatic activity between the liver tissue and the target disease tissue by a method such as Western blotting or immunohistochemical staining, because a required level of the relative difference in enzymatic activity varies depending on the target disease tissue of interest. In one example, when the target disease tissue is the kidney tissue, the protease needs to have 3-fold or more relative difference of enzymatic activity in the kidney tissue from that in the liver tissue and preferably have 10-fold or more such relative difference, more preferably have 100-fold or more such relative difference.

The PEG-modified form of the HGF or the active fragment thereof can be expected to reduce an adverse effect caused by the HGF or the active fragment thereof in the liver tissue, because a chemical modification with PEG can reduce the activity and liver tropism of the HGF or the active fragment thereof. On the other hand, a conjugation of the protease-sensitive peptide selected as above mentioned to the PEG-modified form of the HGF or the active fragment thereof may enable the HGF or the active fragment thereof to be released and exert its bioactivity in the target disease tissue when the protease-sensitive peptide is cleaved by its corresponding protease to liberate the PEG. A fragment of the protease-sensitive peptide cleaved by the corresponding protease may remain in the HGF or the active fragment thereof.

Examples of the protease whose expression is observed in the target disease tissue other than the liver tissue include ADAM17 (Melenhorst W. B. et al., American Journal of Physiology, 2009, Vol. 297, No. 3, p. 781-790), hepatocyte growth factor activator (HGFA), its upstream factor thrombin (David C. J. H. et al., American Journal of Physiology, 2001, Vol. 159, No. 4, p. 1383-1393) and matrix metalloprotease (Junwei Y. et al., The Journal of Clinical Investigation, 2002, Vol. 110, p. 1525-1538) which are reported to be expressed in the kidney of kidney disease patients.

ADAM17 is intracellularly synthesized as an inactive proform and then cleaved by furin into a mature form, that in turn exhibits an activity of cleaving various proteins (Aldo B. et al., Journal of Biological Chemistry, 2003, Vol. 278, p. 25933-25939; and Belen S.J. et al., Journal of Biological Chemistry, 2007, Vol. 282, p. 8325-8331). ADAM17 refers to mature ADAM17 unless otherwise specified.

An endogenous peptide which is previously reported to be cleaved by its corresponding protease, or an artificial peptide prepared by use of a gene recombination technique can be used as the protease-sensitive peptide contained in the PEG-modified form of the HGF or the active fragment thereof. The protease-sensitive peptide may be selected in consideration of a stability, cleavage efficiency in the target tissue and species cross-reactivity. We found that the expressions of ADAM17 and thrombin in the liver tissue is relatively lower than that in the target disease tissue other than the liver tissue. Therefore, the protease-sensitive peptide is preferably an ADAM17-sensitive peptide or a thrombin-sensitive peptide. In this context, the ADAM17-sensitive peptide means a peptide that is cleaved by ADAM17, and the thrombin-sensitive peptide means a peptide that is cleaved by thrombin.

Examples of the endogenous peptide as the ADAM17-sensitive peptide include a peptide derived from TNF-alpha (SPLAQAVRSSSR, SEQ ID NO: 8) and a peptide derived from L-selectin (QETNRSFSK, SEQ ID NO: 10). Examples of the artificial peptide include an artificial ADAM-17 cleavable sequence (PRAAAVKSP, SEQ ID NO: 9) (Caescu C. I. et al., Biochemical Journal, 2009, Vol. 424, p. 79-88). The ADAM17-sensitive peptide is preferably a peptide having the amino acid sequence represented by any of SEQ ID NOs: 8 to 16, more preferably a peptide having the amino acid sequence represented by SEQ ID NO: 9.

Examples of the endogenous peptide as the thrombin-sensitive peptide include a peptide derived from prothrombin (FNPRTFGS, SEQ ID NO: 19) and a peptide derived from HGFA (LRPRIIGG, SEQ ID NO: 20). Examples of the artificial peptide include an artificial thrombin cleavable sequence (TFLTPRGVRLG, SEQ ID NO: 17 and TFPPRSFLG, SEQ ID NO: 18) (Maike G. et al., PLoS ONE, 2012, Vol. 7, p. E31756-31771; and Bo C. et al., The Journal of Neuroscience, 2012, Vol. 32, No. 22, p. 7622-7631). The thrombin-sensitive peptide is preferably a peptide having the amino acid sequence represented by any of SEQ ID NOs: 17 to 20, more preferably a peptide having the amino acid sequence represented by SEQ ID NO: 17.

One or more protease-sensitive peptide sequences can be used. Use of the protease-sensitive peptides differing in cleavage reactivity to the same protease, or the protease-sensitive peptides for different proteases in combination allows a plurality of locations to be chemically modified with PEG or allows cleavage reactivity to be controlled.

The protease-sensitive peptide can be obtained as the protease-sensitive peptide-added HGF or active fragment thereof, for example, by use of a gene recombination technique known in the art by designing a nucleic acid (DNA) sequence encoding the protease-sensitive peptide sequence consecutively with a nucleic acid (DNA) sequence encoding the HGF or the active fragment thereof, and transiently or stably introducing the resulting sequence to cells for expression.

An artificial sequence such as a tag sequence may be added to the protease-sensitive peptide described above for the purpose of purification or the like. Examples of the tag sequence include a 6× His tag, HAT tag, c-Myc tag, FLAG tag, DYKDDDDK tag, Strep tag, HA tag, GST tag and MBP tag. In addition to the protease-sensitive peptide, a sequence such as a protease-sensitive peptide for purification or a spacer sequence for purification may be arbitrarily inserted between the protease-sensitive peptide and such a tag sequence to remove the tag sequence after purification of the protease-sensitive peptide-added HGF or active fragment thereof. In this example, the protease-sensitive peptide-added HGF or active fragment thereof obtained by purification and the removal of the tag sequence may contain a cleaved fragment of the sequence such as the protease-sensitive peptide for purification or the spacer sequence for purification.

A natural amino acid such as cysteine or a non-natural amino acid such as azidophenylalanine may be inserted in the protease-sensitive peptide described above for the purpose of PEG modification or the like. The insertion of the amino acid above mentioned into the protease-sensitive peptide can be carried out, for example, by use of a gene recombination technique known in the art by designing a nucleic acid (DNA) sequence encoding the amino acid to be added consecutively with a nucleic acid (DNA) sequence encoding the protease-sensitive peptide sequence, and transiently or stably introducing the resulting sequence to cells for expression. The insertion site of the amino acid for the purpose of PEG modification or the like is preferably a terminus (amino terminus or carboxyl terminus) of the protease-sensitive peptide.

One to 20 amino acids may be added as a spacer sequence to between the protease-sensitive peptide and the artificial sequence such as the tag sequence for the purpose of purification or the like, or between the protease-sensitive peptide and the amino acid inserted for the purpose of PEG modification or the like, by use of a gene recombination technique known in the art. The protease-sensitive peptide with the spacer sequence between the protease-sensitive peptide and the artificial sequence such as the tag sequence for the purpose of purification or the like or the amino acid inserted for the purpose of PEG modification or the like can be obtained, for example, by use of a gene recombination technique known in the art by cloning a nucleic acid (DNA) sequence encoding the protease-sensitive peptide and adding a nucleic acid (DNA) sequence encoding an arbitrarily selected spacer sequence to be consecutive, and further inserting a nucleic acid (DNA) sequence encoding the artificial sequence such as the tag sequence for the purpose of purification or the like or the amino acid for the purpose of PEG modification or the like thereto to be consecutive, and transiently or stably introducing the resulting sequence to cells for expression. Examples of the amino acid that can be used in the spacer sequence include aspartic acid (D), glutamic acid (E), histidine (H), lysine (K), arginine (R), phenylalanine (F), tyrosine (Y), tryptophan (W), alanine (A), cysteine (C), asparagine (N), glutamine (Q), serine (S), threonine (T), isoleucine (I), leucine (L), norleucine (Nle), methionine (M), valine (V) and tryptophan (W). In this respect, the spacer sequence to be arbitrarily added is preferably added to a terminus of the protease-sensitive peptide. More preferably, the carboxyl terminus of the spacer sequence is added to the amino terminus of the protease-sensitive peptide added to the amino terminus of the HGF or the active fragment thereof, and the artificial sequence such as the tag sequence for the purpose of purification or the like or the amino acid inserted for the purpose of PEG modification or the like is added to the amino terminus of the spacer sequence. The spacer sequence used may be appropriately selected in consideration of the cleavage efficiency of the protease-sensitive peptide, or the control efficiency of the activity of the HGF or the active fragment thereof by PEG modification. PEG

PEG is a highly biocompatible polymer including water-soluble poly(ethylene oxide). It is known that the chemical modification of a protein with PEG imparts clinical usefulness such as improvement in physical stability, heat stability, resistance to proteolytic enzymes, and solubility, decrease in in vivo distribution volume, and improvement in blood circulation to the protein (Inada et al., Journal of Bioact and Compatible Polymers, Vol. 5, 1990, p. 343; Delgado et al., Critical Reviews in Therapeutic Drug Carrier Systems, Vol. 9, 1992, p. 249; and Katre, Advanced Drug Delivery Systems, Vol. 10, 1993, p. 91).

PEG known in the art can be used to prepare the PEG-modified form of HGF or an active fragment thereof. Typically, the PEG has a repeat unit structure “—(CH₂CH₂O)ₙ—,” and a terminal group or a whole structure of the PEG may vary as mentioned later. The PEG may contain, for example, “—CH₂CH₂—O(CH₂CH₂O)ₙ—CH₂CH₂—” and “—(OCH₂CH₂)ₙO—,” depending on the presence or absence of substitution of the terminal oxygen atom. The PEG that is used for preparing the PEG-modified form of the HGF or the active fragment thereof is terminally capped. Specific examples thereof include the PEG capped at one of its ends with a functional group having reactivity (e.g., a maleimide group). The other end of the PEG may be capped or may undergo, for example, modification such as the introduction of a hydroxyl group to enhance hydrophilicity. In using a PEG having a branched structure, at least one end can be capped with the functional group having reactivity such as a maleimide group, and each of the other ends of the branched chain may undergo modification. The PEG activated with a functional group such that the PEG end exhibits covalent binding reactivity with the protease-sensitive peptide-added HGF or active fragment thereof can be used to chemically modify the protease-sensitive peptide-added HGF or active fragment thereof with PEG.

The PEG contained in the PEG-modified form of the HGF or the active fragment thereof is not particularly limited by its structure. The PEG may have a linear structure or a branched structure and preferably has a branched structure from the viewpoint of reducing the liver tropism of the HGF or the active fragment thereof. Examples of the PEG having a branched structure include dibranched PEG, tribranched PEG and tetrabranched PEG, though the number of branches also includes more than four. Among them, the tetrabranched PEG is preferred. More specific examples thereof include a structure dibranched, tribranched or tetrabranched from an end of a linker moiety up to the functional group, a structure where a PEG chain branch further arises from a PEG chain branch arising from an end of a linker moiety up to the functional group, and a structure where PEG chain branches arise from an end of a linker moiety up to the functional group and from the middle of the linker moiety. The PEG chains of the branches may have the same or different number-average molecular weights. Among others, a structure is preferred where two PEG chain branches arise from a linker moiety up to the functional group and two PEG chain branches further arise from the PEG chains of the branches, respectively; and a structure is most preferred where two PEG chain branches arise from a linker moiety up to the functional group and two PEG chain branches further arise from the PEG chains of the branches, respectively, wherein a total of the four PEG chains have the same number-average molecular weight. Examples of the PEG having the most preferred structure include SUNBRIGHT GL4-800MA from Yuka Sangyo Co., Ltd. (NOF Corp.). Preferred examples related to the structure of the PEG and preferred examples related to the number-average molecular weight of the PEG can be arbitrarily combined. Examples thereof include a tetrabranched PEG having a number-average molecular weight of 20000 to 100000, and a PEG having a number-average molecular weight of 70000 to 90000 and having a structure where two PEG chain branches arise from a linker moiety up to the functional group and two PEG chain branches further arise from the PEG chains of the branches, respectively, wherein the all of the four PEG chains have the same number-average molecular weight.

The PEG may be available as a commercial product, or can be synthesized by the ring-opening polymerization of ethylene oxide according to a method known in the art or a method equivalent thereto (Sandler and Karo, Polymer Synthesis, Academic Press, New York, Vol. 3, p. 138-161).

The number-average molecular weight of the PEG contained in the PEG-modified form of the HGF or the active fragment thereof is preferably 20000 to 100000, more preferably 40000 to 100000, further preferably 60000 to 100000, most preferably 70000 to 90000 for the attenuation of HGF activity. It is known that the in vivo half-life extending effect of chemical modification with PEG on a protein or the like correlates with the number-average molecular weight of the PEG (Sundqvist T. et al., Computers and Biomedical Research, 1988, Vol. 21, No. 2, p. 110-116). In general, PEG having a number-average molecular weight of 20000 or larger can be expected to have a sufficient in vivo half-life extending effect.

To chemically modify the protease-sensitive peptide-added HGF or active fragment thereof with PEG, it is necessary to activate one terminus of the PEG. Examples of the activated PEG include PEG activated with a N-hydroxysuccinimide ester, nitrobenzenesulfonate ester, maleimide, o-pyridine disulfide, vinyl sulfone, iodoacetamide, carboxylic acid, azide, phosphine or amine structure at one terminus of the PEG. Such activated PEG may be a commercially available product, or can be synthesized according to a method known in the art. Examples of the PEG activated with each reactive functional group include SUNBRIGHT® (NOF Corp.) series from Yuka Sangyo Co., Ltd. The reactive functional group added to the PEG terminus to exhibit covalent binding reactivity with the protease-sensitive peptide-added HGF or active fragment thereof is simply referred to as a “functional group,” and PEG having such a reactive functional group at its terminus is referred to as “PEG activated with the functional group.” The functional group is appropriately selected depending on a modification site. For example, an amino group can be selectively modified by using a N-hydroxysuccinimide (NETS) ester group as the functional group. Also, a thiol group present in the side chain of a cysteine residue can be selectively modified by using a maleimide group as the functional group. Further, carboxyl groups present in the side chain of glutamic acid and the carboxyl terminus of protein can be modified using an amino group or the like. An azide group contained in a non-natural amino acid such as azidophenylalanine can be selectively modified using triallylphosphine. In these examples, the protease-sensitive peptide-added HGF or active fragment thereof modified with PEG, i.e., the PEG-modified form of HGF or an active fragment thereof, may contain a functional group resulting from the covalent binding reaction between the PEG and the protease-sensitive peptide, and contains, for example, a succinimide group resulted from the covalent binding reaction between a thiol group contained in cysteine and a maleimide group.

Preferred examples of the HGF or the active fragment thereof, preferred examples of the protease-sensitive peptide, and preferred examples of the PEG as described above can be arbitrarily combined.

### Production Method

In the PEG-modified form of the HGF or the active fragment thereof, as mentioned above, the PEG is/are covalently bound to (a) terminus(es) (amino terminus or carboxyl terminus) of the HGF or the active fragment thereof via (a) protease-sensitive peptide(s). Examples of the production method therefor include a method of covalently binding PEG to the protease-sensitive peptide-added HGF or active fragment thereof.

An exemplary method of producing the protease-sensitive peptide-added HGF or active fragment thereof will be described below. The HGF or the active fragment thereof and the protease-sensitive peptide can be expressed as one protein molecule without being separately prepared. The protease-sensitive peptide-added HGF or active fragment thereof can be obtained, for example, by use of a gene recombination technique mentioned later by designing a nucleic acid (DNA) sequence encoding the protease-sensitive peptide sequence consecutively with a nucleic acid (DNA) sequence encoding the HGF or the active fragment thereof, and transiently or stably introducing the resulting sequence to cells for expression. An arbitrary spacer sequence can also be added by designing a nucleic acid (DNA) sequence encoding the spacer sequence such that the spacer sequence is added consecutively between the HGF or the active fragment thereof and the protease-sensitive peptide, or between the protease-sensitive peptide and an artificial sequence such as a tag sequence for the purpose of purification or the like or an amino acid inserted for the purpose of PEG modification or the like.

In the production of the protease-sensitive peptide-added HGF or active fragment thereof, it is preferred to use the protease-sensitive peptide-added HGF or active fragment thereof obtained by expression in a protease-sensitive peptide-added state, for example, by use of a gene recombination technique. In this respect, the protease-sensitive peptide is preferably bound directly to the amino terminus of the HGF or the active fragment thereof. Preferably, the carboxyl terminus of the protease-sensitive peptide is bound directly to the amino terminus of the HGF or the active fragment thereof.

The purification or concentration of the protease-sensitive peptide-added HGF or active fragment thereof can be carried out by use of a method known in the art. The protease-sensitive peptide-added HGF or active fragment thereof can be purified or concentrated, for example, according to a method such as chromatography using ion exchange, gel filtration, a hydrophobic support or an affinity support, or a combination thereof by removing unreacted HGF or active fragment thereof or PEG activated with the functional group, or by-products.

The HGF or the active fragment thereof can also be obtained by use of a method known in the art such as extraction from a tissue, protein synthesis using a gene recombination technique, or biological production using recombinant cells expressing the HGF or the active fragment thereof (or natural cells expressing the HGF). Alternatively, commercially available HGF or active fragment thereof may be used as the HGF or the active fragment thereof.

It is known that the HGF or an active fragment thereof can be expressed using prokaryotic cells or eukaryotic cells. For example, NK1 can be obtained by use of a gene recombination technique known in the art by transiently or stably introducing a nucleic acid (DNA) sequence encoding NK1, an active fragment of HGF, to cells for NK1 expression.

The gene recombination technique can be performed according to a method described in, for example, Molecular Cloning, 2nd ed., Cold Spring harbor Laboratory (1989). An exemplary such technique will be described below.

DNA encoding the HGF or the active fragment thereof is provided. The DNA can be obtained by isolating cDNA by selection from a cDNA library prepared from human tissues or cells, and subjecting the cDNA to a DNA amplification method such as PCR. Alternatively, the DNA can be chemically synthesized using, for example, a DNA synthesizer based on a phosphoramidite method.

The DNA described above is incorporated into an appropriate vector to prepare an expression vector. Such a vector contains elements such as regulatory sequences, necessary for expressing the DNA (if necessary, and for secreting the protein). Specific examples of the elements include a translation start codon and stop codon, a promoter, an enhancer, a terminator, a ribosomal binding site (or a Shine-Dalgarno sequence), a selective marker sequence, and a signal sequence. The necessary elements are inserted in the vector.

A suitable promoter is selected as the promoter according to host cells. Examples of the promoter suitable for cells of Escherichia bacteria which are prokaryotes include trp promoter, lac promoter, recA promoter, and λP_{L} promoter. Examples of the promoter suitable for cells of Bacillus bacteria include SPO1 promoter, SPO2 promoter, and penP promoter. Examples of the promoter suitable for yeast cells include PHOS promoter, PGK promoter, GAP promoter, ADH promoter, and AOX promoter. Examples of the promoter suitable for plant cells include cauliflower mosaic virus (CaMV) promoter. Examples of the promoter suitable for insect cells include P10 promoter and polyhedrin promoter. Examples of the promoter suitable for mammalian cells include promoters of viruses such as Rous sarcoma virus, polyoma virus, fowl-pox virus, adenovirus, bovine papilloma virus, avian sarcoma virus, cytomegalovirus (SMV), simian virus 40 (SV40), and vaccinia virus, metallothionein promoter, and heat shock promoter.

Examples of the selective marker include HIS3 gene, LEU2 gene, TRP1 gene, URA3 gene, dihydrofolate reductase gene (methotrexate (MTX) resistance), ampicillin resistance gene, neomycin resistance gene, and kanamycin resistance gene.

A vector suitable for host cells is selected as the expression vector. For example, plasmids, phages, cosmids, virus vectors, and artificial chromosomes (e.g., BAC and YAC) are vectors usually used. Examples of the vector for a prokaryote include E. coli-derived plasmids, for example, plasmids of pCR series, plasmids of pBR series, and plasmids of pUC series, and Bacillus subtilis-derived plasmids, for example, pUB110, pTP5, and pC194. Examples of the vector for a yeast include yeast-derived plasmids, for example, plasmids of pSH series. Examples of the vector for a plant cell include binary vectors. Examples of the vector for a mammalian cell include commercially available vectors such as pBK-CMV, pcDNA3.1, and pZeoSV (Invitrogen Corp and Stratagene California), and virus vectors (e.g., vectors of adenovirus, adeno-associated virus, pox virus, herpes simplex virus, lentivirus, Sendai virus, vaccinia virus, and SV40).

Examples of the host cells include prokaryotic cells such as E. coli and Bacillus subtilis, and eukaryotic cells such as yeasts, plant cells, and animal cells (e.g., mammalian cells and insect cells). When the host cells are transformed or transfected with the expression vector, an approach known in the art can be used, for example, electroporation, microinjection, cell fusion, DEAE dextran method, calcium phosphate method, or particle gun method.

Examples of the method of binding the protease-sensitive peptide-added HGF or active fragment thereof to the PEG include a method of covalently binding the protease-sensitive peptide-added HGF or active fragment thereof described above to the PEG activated with the functional group, in other words, a method of chemically modifying the protease-sensitive peptide-added HGF or active fragment thereof with the PEG activated with the functional group.

For the chemical modification of the protease-sensitive peptide-added HGF or active fragment thereof described above with the PEG, an amino group, a thiol group, a carboxyl group, an azide group or the like contained in an amino acid contained in the protease-sensitive peptide, or cysteine or a non-natural amino acid (e.g., azidophenylalanine) artificially introduced in the protease-sensitive peptide can be used. For the chemical modification of the protease-sensitive peptide-added HGF or active fragment thereof described above with the PEG, a gene recombination technique known in the art can be used (U.S. Pat. No. 4917888, International Publication No. WO 1987/00056, International Publication No. WO 1999/55377, and Bioorganic & Medicinal Chemistry Letters, Vol. 14, 2004, p. 5743-5745). For example, in artificially insertion of cysteine into the protease-sensitive peptide added to the amino terminus or carboxyl terminus of the HGF or the active fragment thereof, a thiol group present in the side chain of the cysteine residue can be chemically modified in a site-selective manner with the PEG having a maleimide group as a functional group because the thiol group forms neither intramolecular nor intermolecular disulfide bonds. A method of introducing azidophenylalanine and azido-Z-lysine or the like by codon alteration has been reported as a method of inserting a non-natural amino acid to the terminus of the protease-sensitive peptide in the protease-sensitive peptide-added HGF or active fragment thereof (JP Patent Publication (Kokai) No. 2009-207490 A (2009)). An azide group contained in such a non-natural amino acid may be chemically modified in a site-selective manner with the PEG having triallylphosphine as a functional group. Alternatively, the protease-sensitive peptide may be intracellularly labeled with biotin using Biotin-Ligase Expression System known in the art (Avidity LLC) and can be used as a site of chemical modification with the PEG through the use of interaction with avidin.

Among others, a method is preferred which involves artificially insertion of an amino acid (natural or non-natural) that exhibits a selective reactivity with the functional group to the terminus of the protease-sensitive peptide added to the terminus (amino terminus or carboxyl terminus) of the HGF or the active fragment thereof. The insertion of the amino acid (natural or non-natural) that exhibits selective reactivity with the functional group can be carried out by use of the gene recombination technique mentioned above. Particularly, to prepare the PEG-modified form of the HGF or the active fragment thereof, it is more preferred to artificially insert a cysteine residue, an azidophenylalanine residue or an azido-Z-lysine residue, and it is further preferred to artificially insert a cysteine residue. In this example, it is preferred to use the PEG activated with a maleimide group.

The purification or concentration of the PEG-modified form of the HGF or the active fragment thereof can be carried out by use of a method known in the art after chemical modification with the PEG via the protease-sensitive peptide. The PEG-modified form of the HGF or the active fragment thereof can be purified or concentrated, for example, according to a method such as chromatography using ion exchange, gel filtration, a hydrophobic support or an affinity support, or a combination thereof by removing unreacted HGF or active fragment thereof or PEG activated with the functional group, or by-products.

### Medicament

The PEG-modified form of the HGF or the active fragment thereof can not only be expected to have an in vivo half-life extending effect owing to PEG modification but also can exert the bioactivity potentially possessed by the HGF or the active fragment thereof through the cleavage of the protease-sensitive peptide contained in the PEG-modified form in the target disease tissue expressing particular protease, and as such, can be used as an active ingredient for a medicament (e.g., a therapeutic or prophylactic drug for organ fibrosis such as renal fibrosis) with reduced adverse effects for the liver.

The bioactivity of the PEG-modified form of the HGF or the active fragment thereof described above can be conveniently measured using cultured cells. For example, a c-Met phosphorylation-inducing effect, a cell proliferative effect, a cell migration effect and an anti-apoptotic effect which are the pharmacological effects of the natural HGF can be used as indexes (Rubin J. S. et al, The Journal of Biological Chemistry, 2001, Vol. 276, No. 35, p. 32977-32983; Lietha D. et al., The EMBO Journal, 2001, Vol. 20, No. 20, p. 5543-5555; and Liu Y. et al., American Journal of Physiology, 1999, Vol. 277, No. 4, p. 624-633).

The release behavior of the active form, the HGF or the active fragment thereof, from the PEG-modified form of the HGF or the active fragment thereof described above can be conveniently measured, for example, by using SDS electrophoresis in which the molecular weight change between the PEG-modified form and the active form is measured as an index.

The in vivo half-life of the PEG-modified form of the HGF or the active fragment thereof described above can be calculated, for example, by measuring the concentration in blood of the PEG-modified form intravenously, intraperitoneally, subcutaneously or intradermally administered to experimental animals. Particularly, the radiolabeling of the PEG-modified form of the HGF or the active fragment thereof described above conveniently achieves such measurement.

The medicament comprising the PEG-modified form of HGF or an active fragment thereof described above as an active ingredient can be used in the treatment of a disease (acute inflammatory disease, chronic inflammatory disease, acute ischemic disease, chronic ischemic disease and the like) that can exploit the pharmacological effects of the HGF or the active fragment thereof. The medicament can be used in the treatment of, for example, amyotrophic lateral sclerosis, organ fibrosis, diabetes mellitus, spinal cord injury, peritoneal adhesion or neuropathic pain as a specific disease, and further can be used for transplantation therapy, wound healing.

When the protease-sensitive peptide is, for example, an ADAM17-sensitive peptide, the medicament comprising the PEG-modified form of the HGF or the active fragment thereof described above as an active ingredient can be used as a therapeutic or prophylactic agent for a disease (e.g., renal fibrosis) that develops in an organ (e.g., the kidney) having a higher expression level of ADAM17 than that of the liver in which an adverse effect of the HGF or the active fragment thereof is of concern. As for an organ having an equivalent or lower expression level of ADAM17 to or than that of the liver in healthy people, the medicament comprising the PEG-modified form of the HGF or the active fragment thereof described above as an active ingredient can also be used as a therapeutic agent for a disease as long as the disease is related to an organ in which the expression level of ADAM17 elevates due to a lesion and becomes higher than that of the liver.

The medicament described above can be used as a useful therapeutic drug for a mammal (e.g., a mouse, a rat, a hamster, a rabbit, a dog, a cat, a monkey, a cattle, a sheep or a human), particularly, a human. In use of the medicament described above as a useful therapeutic drug for a human, the HGF or the active fragment thereof described above is preferably based on an amino acid sequence derived from human HGF.

In the mode of administration of the medicament described above, the PEG-modified form of the HGF or the active fragment thereof serving as an active ingredient can be orally or parenterally administered, either alone or as a medicament blended with an acceptable carrier. The administration is preferably performed by subcutaneous, intramuscular or intravenous injection.

Examples of the dosage form for the oral administration of the medicament described above include tablets, pills, capsules, granules, syrups, emulsions and suspensions. These dosage forms can be produced by methods known in the art and contain a carrier or an excipient and an optional additive usually used in the pharmaceutical formulation field. Examples of the carrier and the excipient for tablets include lactose, maltose, saccharose, starch and magnesium stearate. Examples of the additive can include binders, disintegrants, preservatives, delayed releasing agents, colorants, flavoring agents, stabilizers, solubilizers, thickeners, and emulsifiers.

Examples of the dosage form for the parenteral administration of the medicament described above include injections, eye drops, ointments, wet dressings, suppositories, transnasal absorption agents, transpulmonary absorption agents, transdermal absorption agents and local sustained-release agents. These dosage forms can be produced by methods known in the art. Solution formulations can be prepared, for example, in a state where the PEG-modified form of the HGF or the active fragment thereof serving as an active ingredient is dissolved in an aseptic aqueous solution for use in an injection or suspended and emulsified in extracts, and embedded in liposomes. Solid formulations can be prepared, for example, as freeze-dried products of the PEG-modified form of the HGF or the active fragment thereof serving as an active ingredient supplemented with an excipient such as mannitol, trehalose, sorbitol, lactose or glucose. Such solid formulations may be further pulverized for use. Such powders may be mixed with polylactic acid, glycolic acid, or the like and solidified for use. Gels can be prepared, for example, by dissolving the PEG-modified form of the HGF or the active fragment thereof serving as an active ingredient in a thickener or a polysaccharide such as glycerin, PEG, methylcellulose, carboxymethylcellulose, hyaluronic acid or chondroitin sulfate. If necessary, these formulations can be supplemented with the additive described above.

The medicament described above can generally be administered at 0.001 mg to 100 mg/kg/dosage, preferably 0.01 mg to 10 mg/kg/dosage, in the range of once a month to once a day, preferably once a month to once a week, though the dose is appropriately determined according to the age and body weight of a patient, a disease to be administered, symptoms, the mode of administration, an administration route, the molecular weight of PEG and the like.

The medicament described above may be blended or used in combination in an appropriate amount with an additional drug to complement or enhance its therapeutic or prophylactic effect or to reduce its dose.

In another aspect, the PEG-modified form of the HGF or the active fragment thereof can be used as a cell differentiation-promoting or -inducing agent comprising the PEG-modified form of the HGF or the active fragment thereof through the use of the morphogenesis-inducing effect of the HGF or the active fragment thereof. Examples of the cell include epithelial cells and neural stem cells.

### EXAMPLES

Hereinafter, our PEG-modified form of the HGF or the active fragment thereof will be specifically described with reference to Examples. However, this disclosure is not limited by these examples.

### Example 1

### Expression of Protease-Sensitive Peptide-Added Human NK1

DNA encoding the human NK1 in which the carboxyl terminus of the protease-sensitive peptide (SEQ ID NO: 9 as an ADAM17-sensitive peptide, SEQ ID NO: 17 as a thrombin-sensitive peptide) was added to the amino terminus of the human NK1, without its secretory signal sequence, and a 6× His tag and a cysteine residue in this order were added to the amino terminus of the protease-sensitive peptide (i.e., CHHHHHH (SEQ ID NO: 4) were added to the protease-sensitive peptide-added human NK1) was prepared by artificial synthesis (FASMAC Co., Ltd.), and inserted to an expression vector Cold-shock promoter/pCold IV (Takara Bio Inc.). A host E. coli SHuffle T7 Express (New England Biolabs, Inc.) was transformed with the prepared plasmid for expression.

Transformation was also performed by a similar method with DNA encoding the human NK1 in which the amino terminus of the protease-sensitive peptide (SEQ ID NO: 9 as an ADAM17-sensitive peptide) was added to the carboxyl terminus of the human NK1, without its signal sequence, and a cysteine residue and 6× His tag in this order were added to the carboxyl terminus of the protease-sensitive peptide (i.e., HHHHHHC (SEQ ID NO: 5) were added to the protease-sensitive peptide-added human NK1). Hereinafter, human NK1 to which the protease-sensitive peptide (i.e., the ADAM17-sensitive peptide or the thrombin-sensitive peptide) and the 6× His tag and the cysteine residue represented by SEQ ID NO: 4 or 5 were added is collectively referred to as the protease-sensitive peptide-added human NK1.

The E. coli described above was spread over LB agar medium containing kanamycin sulfate or carbenicillin and cultured at 37° C. for 18 to 22 hours for the first selective culture. The obtained colony was inoculated to LB medium containing kanamycin sulfate or carbenicillin and cultured for the second selective culture. This culture solution was used as a seed culture solution.

The seed culture solution was cultured in a culture solution containing kanamycin sulfate or carbenicillin, 0.8% glucose, 0.7% glycerol, 2% Bacto-Tryptone, 1% casamino acid, 1% yeast extract, 100 mmol/L potassium phosphate, 10 mmol/L MgSO₄ and 0.5% NaCl. For the culture, a glass flask or a mini jar fermenter was used. After confirmation that the turbidity of the culture solution elevated to an appropriate value, the culture solution was applied to cold shock at 15° C. or lower. At the same time with the cold shock, IPTG was added to the culture solution. 1.5 to 3 hours after the cold shock, the culture was terminated, and the culture solution was harvested and centrifuged to obtain E. coli precipitates expressing the protease-sensitive peptide-added human NK1.

### Example 2

### Purification of Protease-Sensitive Peptide-Added Human NK1

The protease-sensitive peptide-added human NK1 was purified by the following method from the E. coli precipitates obtained in Example 1.

The E. coli precipitates expressing the protease-sensitive peptide-added human NK1 obtained in Example 1 were suspended in 50 mmol/L Tris-HCl, 5 mmol/L EDTA, 150 mmol/L NaCl, 10 mmol/L CaCl₂, and 10 mmol/L MgCl₂ and disrupted using a high-pressure tabletop homogenizer (GEA Niro Soavi), and then, the homogenate was purified by centrifugation.

The centrifugally purified E. coli homogenate was passed through a heparin support (HiTrapHeparin; GE Healthcare Japan Corp.) equilibrated in advance with PBS(−) so that the protease-sensitive peptide-added human NK1 was bound to the heparin support. Subsequently, a buffer in which a concentration of NaCl contained in PBS(−) varied in the concentration range of 150 mmol/L to 2000 mmol/L was passed therethrough in the ascending order of the NaCl concentration to obtain each elution fraction. Each elution fraction was subjected to SDS electrophoresis to confirm an elution fraction of the protease-sensitive peptide-added human NK1.

Next, the elution fraction of the protease-sensitive peptide-added human NK1 obtained by heparin resin purification was passed through a nickel support (cOmplete® (Roche Diagnostics K.K.) His-Tag Purification Resin; Roche) equilibrated in advance with PBS(−) containing 300 mmol/L NaCl so that the protease-sensitive peptide-added human NK1 was bound to the resin. Subsequently, a buffer obtained by adding imidazole (concentration range: 0 mmol/L to 500 mmol/L) to PBS(−) containing 500 mM NaCl was passed therethrough in the ascending order of the imidazole concentration to obtain each elution fraction. Each elution fraction was subjected to SDS electrophoresis to confirm an elution fraction of the protease-sensitive peptide-added human NK1. The obtained elution fraction of the protease-sensitive peptide-added human NK1 was concentrated using a centrifugal ultrafiltration device (Amicon Ultra-15, MWCO=10000; Merck Millipore) to obtain the protease-sensitive peptide-added human NK1.

### Example 3

### Synthesis of Prodrug of Human NK1

Human NK1 in which one molecule of PEG was covalently bound to the amino-terminal cysteine residue or carboxyl-terminal cysteine residue of one molecule of the protease-sensitive peptide-added human NK1 via the protease-sensitive peptide (hereinafter, also referred to as a prodrug of human NK1) was synthesized by the following method.

The vehicle of the protease-sensitive peptide-added human NK1 obtained by Example 2 was replaced with a 100 mmol/L phosphate buffer solution (pH 6.0) containing 300 mmol/L NaCl and 2 mmol/L EDTA, and PEG activated with the functional group was added to the protease-sensitive peptide-added human NK1 at a molar ratio of 40 times. The PEG was covalently bound to the protease-sensitive peptide-added human NK1 by overnight incubation at 25° C. to synthesize a prodrug of human NK1. The PEG activated with the functional group used was SUNBRIGHT GL2-400MA (dibranched type, number-average molecular weight: 40000, Yuka Sangyo Co., Ltd.), SUNBRIGHT ME-400MA (linear type, number-average molecular weight: 40000, Yuka Sangyo Co., Ltd.) and SUNBRIGHT GL4-800MA (tetrabranched type, number-average molecular weight: 80000, Yuka Sangyo Co., Ltd.). The human NK1 with the ADAM17-sensitive peptide (SEQ ID NO: 9) added to the amino terminus was chemically modified with these three types of PEG (hereinafter, collectively referred to as an ADAM17-cleavable prodrug of human NK1, and described together with the structure and number-average molecular weight of the PEG used in the modification). The human NK1 with the ADAM17-sensitive peptide (SEQ ID NO: 9) added to the carboxyl terminus was chemically modified with the tetrabranched PEG having a number-average molecular weight of 80000. The human NK1 with the thrombin-sensitive peptide (SEQ ID NO: 17) added to the amino terminus was chemically modified with the dibranched PEG having a number-average molecular weight of 40000 (hereinafter, referred to as a thrombin-cleavable prodrug of human NK1).

Next, the reaction solution containing the prodrug of human NK1 was diluted 10-fold with NaCl-free PBS(−) and then passed through an ion-exchange support (SP-Sepharose 6 Fast Flow; GE Healthcare Japan Corp.) to remove unreacted PEG activated with the functional group from the solution. The prodrug of human NK1 adsorbed onto the ion-exchange support was eluted with PBS(−) containing 1.0 mol/L NaCl and then passed through a heparin support (Heparin-Sepharose Fast Flow; GE Healthcare Japan Corp.), and the flow-through solution was collected and concentrated using a centrifugal ultrafiltration device (Amicon Ultra-4, MWCO=30000; Merck Millipore) to obtain a prodrug of human NK1 which was the protein of interest.

### Example 4

### Measurement of Purity of Prodrug of Human NK1 and Contamination Rate of Protease-Sensitive Peptide-Added Human NK1

The purity of the prodrug of human NK1 and the contamination rate of the precursor, the protease-sensitive peptide-added human NK1 having no chemical modification with PEG, were measured by reverse-phase chromatography using high-performance liquid chromatography.

A reverse-phase column (Intrada WP-RP; Imtakt Corp.) was connected to high-performance liquid chromatography (LC-10AD system; Shimadzu Corp.), and the ADAM17-cleavable prodrug of human NK1 (3 types) obtained in Example 3 was injected thereto. A peak was detected at a wavelength of 280 nm while the acetonitrile ratio of distilled water containing 0.1% trifluoroacetic acid (acetonitrile: 0%) was increased to 100% over time. A peak around a relative retention time of 0.65 minutes with respect to the prodrug of human NK1 that appeared as a main peak was regarded as the protease-sensitive peptide-added human NK1. The purity of the prodrug of human NK1 and the contamination rate of the protease-sensitive peptide-added human NK1 were calculated.

As a result, the purity of the ADAM17-cleavable prodrug of human NK1 chemically modified with the dibranched PEG having a number-average molecular weight of 40000 was 98.9% (contamination rate of the protease-sensitive peptide-added human NK1: 1.1%). The purity of the ADAM17-cleavable prodrug of human NK1 chemically modified with the linear PEG having a number-average molecular weight of 40000 was 97.3% (contamination rate of the protease-sensitive peptide-added human NK1: 2.7%). The purity of the ADAM17-cleavable prodrug of human NK1 chemically modified with the tetrabranched PEG having a number-average molecular weight of 80000 was 97.9% (contamination rate of the protease-sensitive peptide-added human NK1: 2.1%). Thus, we confirmed that all the prodrugs of human NK1 were able to be obtained with high purity.

### Example 5

### Measurement of Amount of ADAM17 Activity Exerted in Kidney Disease Model Mouse Tissue

The amount of ADAM17 activity exerted in kidney disease model mouse tissues was measured using an ADAM17 activity measurement kit (SENSOLYTER 520 TACE; Anaspec Inc.). The measurement was performed in duplicate.

Mice with unilateral ureteral obstruction (hereinafter, referred to as UUO mice) were prepared as kidney disease model mice. The ureter of each male mouse of ICR strain (Charles River Laboratories Japan, Inc.) was ligated with a silk thread. The incision site was sutured. After raising for 1, 7, 10 and 14 days, the kidney and the liver of the mouse were harvested. Untreated mice (hereinafter, referred to as normal mice) were raised for 0 days and 14 days, and then, the kidney and the liver of each mouse were harvested. A vehicle obtained by adding 0.1% Triton-X100 to Component C included in SENSOLYTER 520 TACE (Anaspec Inc.) was added to tissue pieces, and the tissue was disrupted using a handy microhomogenizer (Physcotron). The disrupted tissue was left standing at 4° C. or on ice for 15 minutes and centrifuged at 2000×g at 4° C. for 15 minutes to obtain a supernatant.

The total protein level of the centrifugation supernatant was measured using Protein Assay (Bio-Rad Laboratories, Inc.) reagent.

The ADAM17 activity in the centrifugal supernatant was detected by measuring fluorescence at 535 nm upon excitation at 485 nm to calculate ADAM17 activity per μg of the protein.

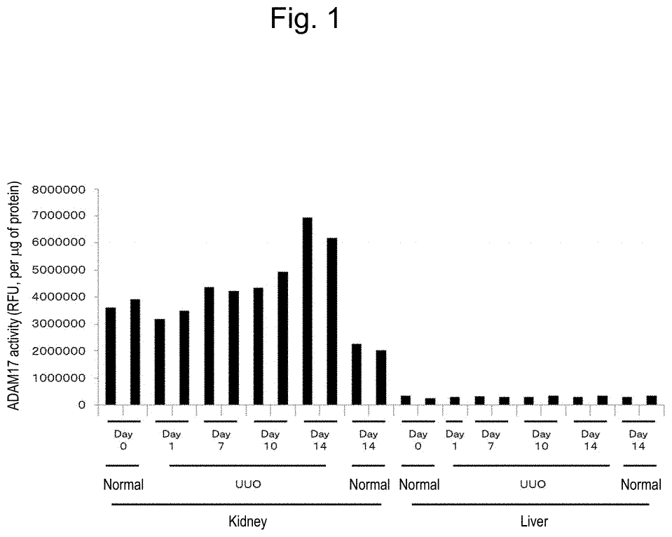

The results are shown in FIG. 1. The ordinate depicts the ADAM17 activity per μg of the protein in each sample. “Day 0,” “Day 1,” “Day 7,” “Day 10” and “Day 14” on the abscissa represent the tissues of the mice raised for 0, 1, 7, 10, and 14 days, respectively. “Normal” represents the normal mice, and “UUO” represents the UUO mice.

Both the UUO mice and the normal mice exhibited ADAM17 activity in the kidney tissues exceeding that in the liver tissues. The activity of ADAM17 was increased with increase in the number of days after ureteral obstruction, indicating that the ADAM17 activity is increased in the kidney tissues with an advanced kidney disease compared to the liver tissues and normal kidney tissues. Thus, ADAM17 was found to be useful as a target molecule for use in the activity control of the prodrug of the HGF or the active fragment thereof.

### Example 6

### Measurement of ADAM17 Protein Expression Level in Kidney Disease Model Mouse Tissue

The expression level of the ADAM17 protein in kidney disease model mouse tissues was evaluated by Western blotting. The evaluation was performed in duplicate.

The kidney and the liver were harvested from each of UUO mice prepared in the same way as in Example 5 on 7 and 14 days after ureteral obstruction for the UUO mice and normal mice on the day when the ureteral obstruction of the UUO mice was carried out for the normal mice (i.e., raised for 0 days). A vehicle obtained by adding 0.1% Triton-X100 to Component C included in SENSOLYTER® (Anaspec Inc.) 520 TACE (Anaspec Inc.) was added to tissue pieces, and the tissue was disrupted using a handy microhomogenizer (Physcotron). The disrupted tissue was left standing at 4° C. or on ice for 15 minutes and centrifuged at 2000×g at 4° C. for 15 minutes to obtain a supernatant.

Western blotting was performed using the obtained supernatant. An ADAM17 proform and mature ADAM17 were detected using Anti-ADAM17 antibody-Activation site (Abcam plc) as a primary antibody and anti-Rab IgG-HRP antibody (Cell Signaling Technology, Inc.) as a secondary antibody. GAPDH was detected using Human/Mouse/Rat GAPDH/G3PH Antibody (R&D Systems, Inc.) as a primary antibody and donkey anti goat IgG-HRP (Santa Cruz Biotechnology, Inc.) as a secondary antibody.

Images obtained with ChemiDoc® (Bio-Rad Laboratories, Inc.) XRS+ System (Bio-Rad Laboratories, Inc.) were analyzed. A value was calculated by dividing the band intensity of the ADAM17 proform or the mature ADAM17 by the band intensity of GAPDH, and used as an index for the expression level of the ADAM17 protein in the tissue.

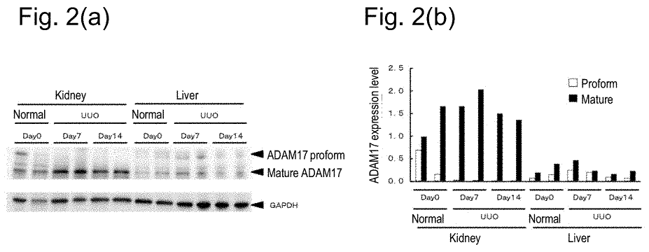

The results are shown in FIG. 2. FIG. 2(a) shows the Western blotting image of each sample. FIG. 2(b) shows a mean calculated from the expression level of the ADAM17 protein in the tissue calculated from the band intensity of each sample. “Day 0,” “Day 7” and “Day 14” in the drawing represent the mice raised for 0, 7 and 14 days, respectively. “Normal” represents the normal mice, and “UUO” represents the UUO mice. The ordinate of FIG. 2(b) depicts the expression level of the ADAM17 protein in the tissue.

Decrease in the expression level of the ADAM17 proform protein and increase in the expression level of the mature ADAM17 protein exhibiting ADAM17 activity were observed over time after ureteral obstruction in the kidney tissues.

These results demonstrated that the expression level of the mature ADAM17 protein is increased in the kidney tissues with an advanced kidney disease compared to the liver tissues or normal kidney tissues.

Thus, ADAM17 was found to be useful as a target molecule for use in the activity control of the prodrug of the HGF or the active fragment thereof.

### Example 7

### Evaluation of Active Form Release by Protease Treatment of Prodrug of Human NK1

The amount of human NK1 (active form) released by the protease treatment of the prodrug of human NK1 was evaluated by SDS electrophoresis.

Each prodrug of human NK1 (three ADAM17-cleavable prodrugs of human NK1 and one thrombin-cleavable prodrug of human NK1) obtained in Example 3 was diluted 10-fold with Component C included in SENSOLYTE 520 TACE (Anaspec Inc.). For the ADAM17-cleavable prodrug of human NK1, ADAM17 (TACE, His Tag, Human Recombinant; Calbiochem) was added at a reaction molar ratio of prodrug of human NK1:ADAM17=5:1. For the thrombin-cleavable prodrug of human NK1, thrombin (Thrombin from human plasma; Sigma-Aldrich Co. LLC) was added at a reaction molar ratio of prodrug of human NK1:thrombin=1:10. The following operation was similarly performed on the prodrug of human NK1 non-supplemented with the corresponding protease as an experimental control. After incubation at 37° C. for 1 hour, the reaction solution was subjected to TCA precipitation and SDS electrophoresis, and a molecular weight position of a band obtained by CBB staining was confirmed.

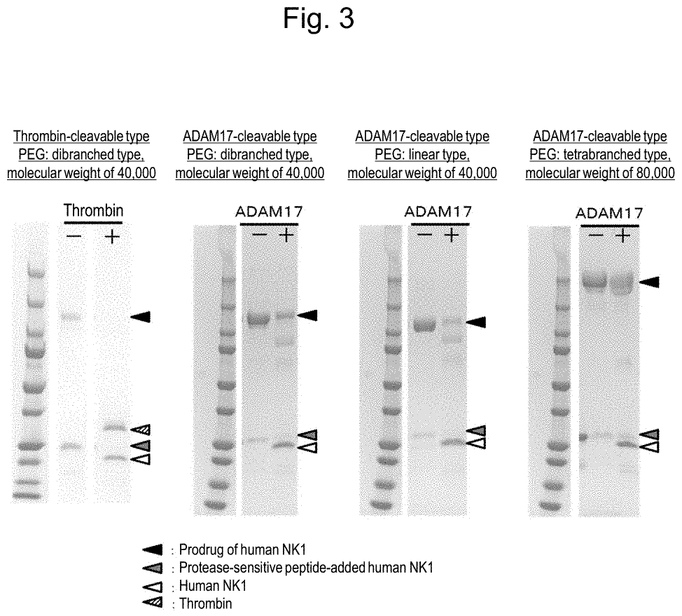

The results are shown in FIG. 3. In the drawing, “-” represents a sample non-supplemented with protease, and “+” represents a sample supplemented with protease. “Thrombin-cleavable type” represents the thrombin-cleavable prodrug of human NK1, and “ADAM17-cleavable type” represents the ADAM17-cleavable prodrug of human NK1. The molecular weight refers to a number-average molecular weight.

The treatment of each prodrug of human NK1 with the corresponding protease (ADAM17 or thrombin) was found to cleave the protease-sensitive peptide contained in the prodrug of human NK1 so that the active form, human NK1, was released.

### Example 8

### Evaluation of Activity Attenuation by Chemical Modification of NK1 With PEG

The degree of attenuation of the HGF activity of the ADAM17-cleavable prodrug of human NK1 was evaluated by In-Cell ELISA using the amount of phosphorylation induced for HGF receptor present on the cell surface of a human lung epithelial cell line A549 as an index.

The A549 cells were suspended in MEM medium containing 10% FCS (Nacalai Tesque, Inc.), seeded at a density of 1.5×10⁴ cells/well on a 96-well plate for imaging (Becton, Dickinson and Company), and cultured all night and all day. After reaching 70% confluency of the cells, the medium was replaced with serum-free MEM medium (Nacalai Tesque, Inc.), and the cells were cultured for 16 hours or longer into a serum-starved state. For the cells in the serum-starved state, the prodrug of human NK1 treated with ADAM17 or the prodrug of human NK1 untreated with ADAM17 in Example 7 was added, and reacted at 37° C. for 10 minutes. In this Example, the prodrug of human NK1 used was the ADAM17-cleavable prodrug of NK1 (3 types) obtained by amino-terminal modification in Example 3.

The cells were fixed in PBS(−) containing 4% formalin. Subsequently, the cell membranes were permeabilized with PBS(−) containing 0.3% Triton-X and 0.6% hydrogen peroxide. Then, the cells were blocked with 10% BSA. For the cells thus blocked, anti-pYcMet antibody (Cell Signaling Technology, Inc.) as a primary antibody and anti-Rab IgG-HRP antibody (Cell Signaling Technology, Inc.) as a secondary antibody were added, and reacted, followed by the addition of 1× QuantaRed Enhanced Chemifluorescent HRP Substrate (Thermo Fisher Scientific, Inc.). After incubation at room temperature, fluorescence intensity (RFU) at a wavelength of 590 nm was measured using a plate reader (Envision; PerkinElmer, Inc.) and used as the induced amount of HGF receptor phosphorylation.

Using the induced amount of HGF receptor phosphorylation, EC50 of a concentration reaction curve of the sample untreated with ADAM17 was calculated using Prism 4 (GraphPad, 4-parameter approximation). Next, a value (fold-change of an activity (hereinafter, referred to as y)) was calculated as the degree of HGF activity attenuation by PEG modification, by dividing the EC50 (nM) of the sample untreated with ADAM17 by a concentration (nM) of the sample treated with ADAM17 at which the same signal intensity as that for the EC50 value of the sample untreated with ADAM17 was attained.

Next, the CBB staining images obtained in Example 7 were analyzed using ChemiDoc XRS+ system (Bio-Rad Laboratories, Inc.), and the cleavage efficiency (hereinafter, referred to as w) of the prodrug of human NK1 treated with ADAM17 was calculated according to formula (1) below. The HGF activity (hereinafter, preNK1) of the protease-sensitive peptide-added human NK1 (i.e., the precursor contained in the sample non-supplemented with ADAM17 due to poor purification), the HGF activity (hereinafter, Pro) of the prodrug of human NK1, the HGF activity (hereinafter, Pro-cut) of the prodrug of human NK1 cleaved by ADAM17 contained in the sample supplemented with ADAM17, and the HGF activity (hereinafter, Pro-noncut) of the prodrug of human NK1 uncleaved by ADAM17 (i.e., the prodrug that remained without being cleaved) were defined according to formulas (2) to (5), respectively, using the purity (%; hereinafter, referred to as v) of the prodrug of NK1 obtained in Example 4. In this respect, the relative activity (%) of the prodrug of NK1 was indicated by as (algebra) when the activity of the protease-sensitive peptide-added human NK1 was defined as 100.

Cleavage efficiency (%, w) of the prodrug of human NK1=100−(Band intensity of the prodrug of human NK1 in the sample supplemented with ADAM17/Band intensity of the prodrug of human NK1 in the sample non-supplemented with ADAM17)×100 (1)

Activity (preNK1) of the protease-sensitive peptide-added human NK1 contained in the sample non-supplemented with ADAM17=(100−v)/100×1 (2)

Activity (Pro) of the prodrug of human NK1 contained in the sample non-supplemented with ADAM17=(v/100)×(x/100) (3)

Activity (Pro-cut) of the cleaved prodrug of human NK1 contained in the sample supplemented with ADAM17=(v/100)×(w/100)×1 (4)

Activity (Pro-noncut) of the uncleaved prodrug of human NK1 contained in the sample supplemented with ADAM17=(v/100)×(100−w)/100×(x/100) (5)

In this context, the fold-change of the activity (y) can be described according to formula (6) using the expressions defined according to formulas (1) to (5).

Fold-change of the activity (y)=(preNK1+Pro-cut+Pro-noncut)/(preNK1+Pro) (6)

Formula (6) was resolved as to algebra x to obtain formula (7) below. The actually measured values of v, w and y were substituted into formula (7) to calculate the relative activity (%) x of the prodrug of NK1.

x={10⁴×(100−v)+10²×v×w×10⁴×y×(100−v)}/{v×(10²×y)−v×(100−w)} (7)

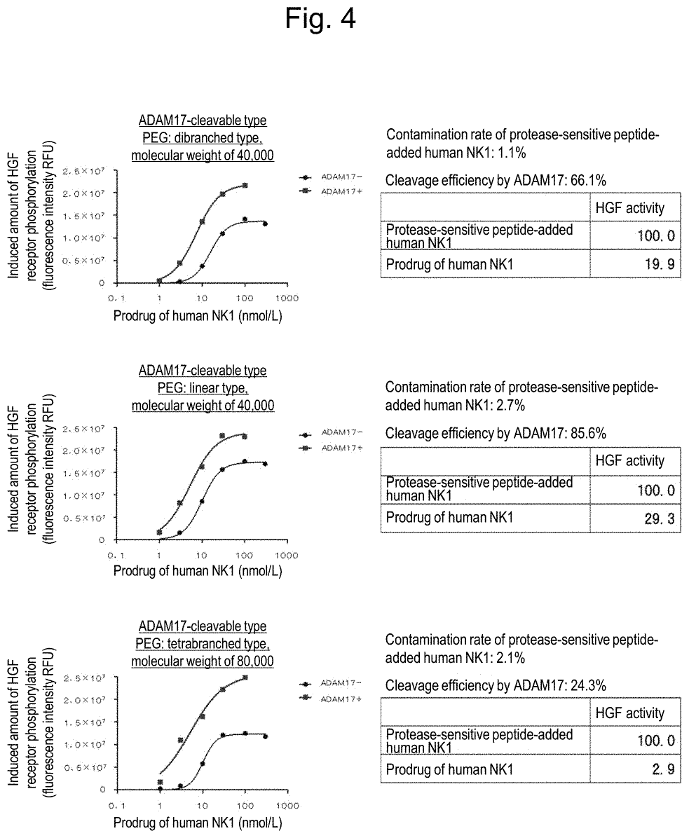

The results are shown in FIG. 4. The ordinate depicts the fluorescence intensity (RFU) that indicates the induced amount of HGF receptor phosphorylation. The abscissa depicts the treatment concentration (nmol/L) of the prodrug of human NK1. In the drawing, “ADAM17−” represents the sample of the prodrug of human NK1 untreated with ADAM17, and “ADAM17+” represents the sample of the prodrug of human NK1 treated with ADAM17. “ADAM17-cleavable type” represents the ADAM17-cleavable prodrug of human NK1. The molecular weight refers to a number-average molecular weight.

Among all the ADAM17-cleavable prodrugs of human NK1 chemically modified with PEG, the prodrug of human NK1 untreated with ADAM17 (ADAM17−) exhibited lower activity than that of the prodrug of human NK1 treated with ADAM17 (ADAM17+), indicating that the HGF activity was attenuated due to the chemical modification with PEG. As compared with the protease-sensitive peptide-added human NK1 having no chemical modification with PEG, the HGF activity of the ADAM17-cleavable prodrug of human NK1 chemically modified with the dibranched PEG having a number-average molecular weight of 40000 was attenuated by 80.1%, the HGF activity of the ADAM17-cleavable prodrug of NK1 chemically modified with the linear PEG having a number-average molecular weight of 40000 was attenuated by 70.7%, and the HGF activity of the ADAM17-cleavable prodrug of NK1 chemically modified with the tetrabranched PEG having a number-average molecular weight of 80000 was attenuated by 97.1%. The HGF activity of the prodrug of human NK1 chemically modified with the tetrabranched PEG having a number-average molecular weight of 80000 among the three types of PEG was most attenuated and was 1/30 of the HGF activity of human NK1 released by ADAM17 treatment.

### Example 9

### Evaluation of Tissue-Selective Release of Active Form From Prodrug of Human NK1

The amount of human NK1 (active form) released by the exposure of the prodrug of human NK1 to a tissue homogenate was evaluated by SDS electrophoresis. In this Example, the amino-terminally modified ADAM17-cleavable prodrug of human NK1 was used which was obtained by modification with the tetrabranched PEG having a number-average molecular weight of 80000 in Example 3. The evaluation was performed in duplicate or triplicate.

UUO mice were prepared in the same way as in Example 5 using male mice of C57BL/6J strain (Charles River Laboratories Japan, Inc.). After ureteral obstruction and raising for 16 days, the kidney and the liver were harvested from each of the UUO mice and normal mice.

To each of the harvested mouse kidney and liver, a buffer solution (pH 7) containing 50 mmol/L Tris-HCl, 10 mmol/L CaCl₂ and 0.05% Brij35 was added, and a supernatant of a tissue homogenate was obtained in the same way as in Example 6. The protein level in the supernatant of the tissue homogenate was measured using Protein Assay (Bio-Rad Laboratories, Inc.).

The ADAM17-cleavable prodrug of human NK1 was added to the supernatant of each tissue homogenate, and incubated at 37° C. for 30 minutes.

Western blotting was performed using the sample incubated above. HGFα (B-3) mouse monoclonal IgG (Santa Cruz Biotechnology, Inc.) was used as a primary antibody, and Peroxidase-conjugate AffiniPure Donkey Anti-Mouse IgG (Jackson ImmunoResearch Laboratories, Inc.) was used as a secondary antibody. Since the band of the prodrug of NK1 was not able to be detected by this method, bands of the protease-sensitive peptide-added human NK1 (i.e., the precursor contaminated due to a poor purification) contained in the prodrug of human NK1 and human NK1 released from the ADAM17-cleavable prodrug of human NK1 were detected and used as an index in evaluation.

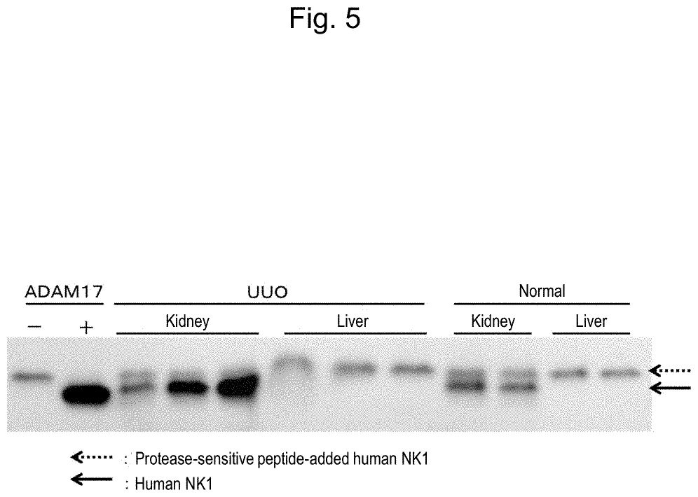

The Western blotting image of each sample is shown in FIG. 5. The band of the active form, human NK1, was detected in the sample of the ADAM17-cleavable prodrug of human NK1 admixed with the kidney homogenate, and its band intensity was higher than that of the protease-sensitive peptide-added human NK1 (i.e., the precursor contaminated due to a poor purification) contained in the prodrug of human NK1, indicating that this human NK1 was the active form released from the prodrug of human NK1. The band intensity of human NK1 was higher in the UUO kidney homogenate having high ADAM17 activity, indicating that a larger amount of human NK1 was released. In contrast, no band of the active form human NK1 was detected in the ADAM17-cleavable prodrug of human NK1 admixed with the liver homogenate. Thus, the ADAM17-cleavable prodrug of human NK1 was found to release the active form, human NK1, in a kidney tissue-selective manner.

Thus, the ADAM17-cleavable prodrug of human NK1 was found to reduce HGF activity in the liver tissues, exhibit higher HGF activity in highly ADAM17-expressing kidney tissues than that in the liver tissues, be able to be expected to have drug efficacy in the kidney tissues, and be capable of reducing adverse effects in the liver tissues.

### Example 10

### In Vivo Activity Measurement of Prodrug of Human NK1

The in vivo tissue-selective induced amount of c-Met phosphorylation by the ADAM17-cleavable prodrug of human NK1 was evaluated by Western blotting using tissue homogenates of mice given the ADAM17-cleavable prodrug of human NK1 or the protease (ADAM17)-sensitive peptide-added human NK1. In this Example, the amino-terminally modified ADAM17-cleavable prodrug of human NK1 was used which was obtained by chemical modification with the dibranched PEG having a number-average molecular weight of 40000 in Example 3, and also, the protease (ADAM17)-sensitive peptide-added human NK1 was used which was obtained in Example 2.

UUO mice were prepared in the same way as in Example 5. Fourteen days after ureteral obstruction, the protease (ADAM17)-sensitive peptide-added human NK1 (PEG-unmodified) or the prodrug of human NK1 having the dibranched PEG having a number-average molecular weight of 40000 was administered at a dose of 400 μg/kg from the tail vein. The following operation was similarly performed on the normal mice and UUO mice given a vehicle (PBS(−) containing 0.3 mol/L NaCl) for the prodrug of human NK1 as an experimental control. The kidney and the liver were harvested from each mouse at 0.18 hours after administration.

The mouse kidney and liver were each disrupted using a tabletop multi-sample cell disruption apparatus (Shake Master Neo; Bio-Medical Science Co., Ltd.). RIPA Buffer supplemented with a phosphatase inhibitor and a protease inhibitor was added to each homogenate, and the mixture was left standing on ice for 2 hours and then centrifuged to obtain a supernatant. The protein level was measured in the same way as in Example 8.

Western blotting was performed using the supernatant. Total c-Met was detected using anti-total cMet antibody (Cell Signaling Technology, Inc.) as a primary antibody and anti-Ms IgG-HRP antibody (Jackson ImmunoResearch Laboratories, Inc.) as a secondary antibody. Phosphorylated c-Met was detected using anti-pYcMet antibody (Cell Signaling Technology, Inc.) as a primary antibody and anti-Rab IgG-HRP antibody (Cell Signaling Technology, Inc.) as a secondary antibody.

Images taken using ChemiDoc XRS+ system (Bio-Rad Laboratories, Inc.) were analyzed. A value was calculated by dividing the band intensity of phosphorylated c-Met by the band intensity of Total c-Met. From the calculated value, a relative value was calculated with the value in the kidney of the UUO mouse of vehicle administration group defined as 1, and used as an index for the induced amount of HGF receptor phosphorylation in evaluation.

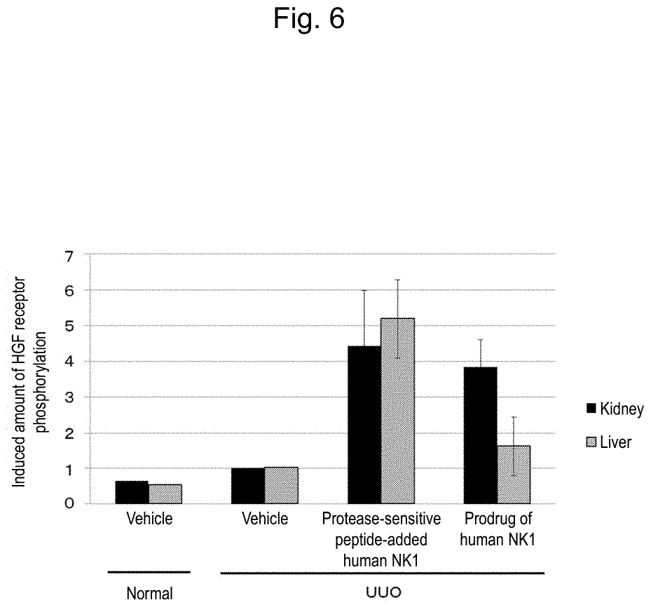

The results are shown in FIG. 6. The ordinate depicts the value of the induced amount of HGF receptor phosphorylation. On the abscissa, “Normal+Vehicle” represents the vehicle administration group of the normal mice, “UUO+Vehicle” represents the vehicle administration group of the UUO mice, “UUO+Protease-sensitive peptide-added human NK1” represents the protease-sensitive peptide-added human NK1 administration group of the UUO mice, and “UUO+Prodrug of human NK1” represents the prodrug-of-human NK1 administration group of the UUO mice.

In the UUO mouse kidney tissues, the induced amount of HGF receptor phosphorylation was equivalent between the protease-sensitive peptide-added human NK1 administration group and the prodrug-of-human NK1 administration group. In contrast, for the liver tissues, the induced amount of HGF receptor phosphorylation by the prodrug of human NK1 was attenuated by 69% as compared with the protease-sensitive peptide-added human NK1. Thus, the prodrug of human NK1 was found to selectively exert the bioactivity in a target disease tissue (i.e., a kidney tissue highly expressing ADAM17) in vivo and reduce the liver tropism potentially possessed by the protease-sensitive peptide-added human NK1.

### Example 11

### Comparison of Activity Control of Prodrug of Human NK1 Depending on Difference in PEG Modification Site

Influence on HGF activity control depending on a difference in the positions of PEG modification of the protease-sensitive peptide-added human NK1 with the ADAM17-sensitive peptide added to the amino terminus obtained in Example 2 was evaluated by In-Cell ELISA using the induced amount of phosphorylation of HGF receptor present on the cell surface of a human lung epithelial cell line A549 as an index. In this Example, the ADAM17-cleavable prodrug of human NK1 was used which was obtained by the chemical modification of the amino terminus of human NK1 with the dibranched PEG having a number-average molecular weight of 40000 via the ADAM17-sensitive peptide (SEQ ID NO: 9) in Example 3 (hereinafter, referred to as amino-terminally modified prodrug of human NK1), and also, the ADAM17-cleavable prodrug of human NK1 was used which was obtained by the chemical modification of the carboxyl terminus of human NK1 with the dibranched PEG having a number-average molecular weight of 40000 via the ADAM17-sensitive peptide (SEQ ID NO: 9) in Example 3 (hereinafter, referred to as carboxyl-terminally modified prodrug of human NK1).

The A549 cells were suspended in MEM medium containing 10% FCS (Nacalai Tesque, Inc.), seeded at a density of 1.5×10⁴ cells/well to a 96-well plate for imaging (Becton, Dickinson and Company), and cultured all night and all day. After reaching 70% confluency of the cells, the medium was replaced with serum-free MEM medium (Nacalai Tesque, Inc.), and the cells were cultured for 16 hours or longer into a serum-starved state. For the cells in the serum-starved state, the prodrug of human NK1 or the protease-sensitive peptide-added human NK1 was added, and reacted at 37° C. for 10 minutes.

The cells were fixed in PBS(−) containing 4% formalin. Subsequently, the cell membranes were permeabilized with PBS(−) containing 0.3% Triton-X and 0.6% hydrogen peroxide. Then, the cells were blocked with 10% BSA. To the cells thus blocked, anti-pYcMet antibody (Cell Signaling Technology, Inc.) as a primary antibody and anti-Rab IgG-HRP antibody (Cell Signaling Technology, Inc.) as a secondary antibody were added, and reacted, followed by the addition of 1× QuantaRed Enhanced Chemifluorescent HRP Substrate (Thermo Fisher Scientific, Inc.). After incubation at room temperature, fluorescence intensity (RFU) at a wavelength of 590 nm was measured using a plate reader (Envision; PerkinElmer, Inc.) and used as the induced amount of HGF receptor phosphorylation.

Using the induced amount of HGF receptor phosphorylation, a relative value for each prodrug of human NK1 with the protease-sensitive peptide and PEG added to the amino terminus or the carboxyl terminus of human NK1 was calculated when the value for the corresponding protease-sensitive peptide-added human NK1 unmodified with PEG was defined as 100.

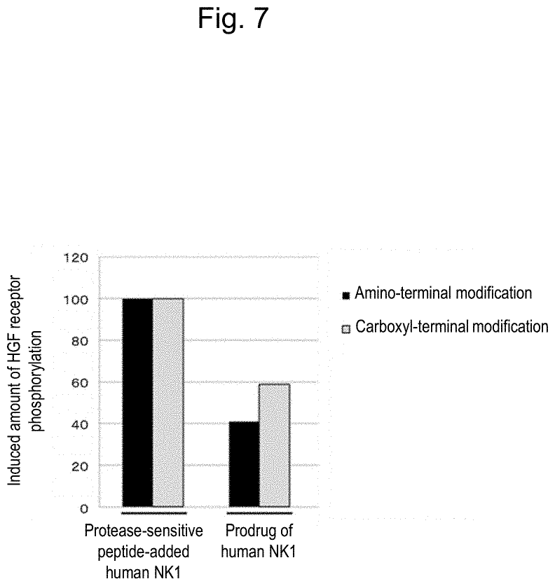

The results are shown in FIG. 7. The ordinate depicts the value of the induced amount of HGF receptor phosphorylation, which is indicated as a relative value when the value from the treatment with the corresponding protease-sensitive peptide-added human NK1 is defined as 100. The legend “Amino-terminal modification” represents the sample in which the protease-sensitive peptide and PEG were added to the amino terminus of the human NK1, and the legend “Carboxyl-terminal modification” represents the sample in which the protease-sensitive peptide and PEG were added to the carboxyl terminus of the human NK1.

The induced amount of HGF receptor phosphorylation (relative value) by the prodrug of human NK1 was 41 for the amino-terminally modified prodrug of human NK1 and, in contrast, was 58 for the carboxyl-terminally modified prodrug of human NK1. The activity of the amino-terminally modified prodrug of human NK1 was more attenuated by chemical modification with PEG as compared with the carboxyl-terminally modified prodrug of human NK1. Thus, our PEG-modified form of HGF or an active fragment thereof was found to be able to more attenuate the activity of the HGF or the active fragment thereof through modification on the amino terminus thereof.

### INDUSTRIAL APPLICABILITY

Our polyethylene glycol-modified form of a hepatocyte growth factor or an active fragment thereof can selectively release the hepatocyte growth factor or the active fragment thereof in a target disease tissue and therefore exhibits high activity in the target disease tissue, while the polyethylene glycol-modified form can be used as a medicament with reduced adverse effects caused by the hepatocyte growth factor or the active fragment thereof in liver tissues.

### Free Text of Sequence Listing

SEQ ID NO: 1: Amino acid sequence of human HGF containing no secretory signal sequence
SEQ ID NO: 2: Amino acid sequence of human NK1 containing no secretory signal sequence
SEQ ID NO: 3: Secretory signal sequence derived from human HGF
SEQ ID NO: 4: Amino acid sequence of a cysteine residue and 6× His tag added to the amino terminus of human NK1
SEQ ID NO: 5: Amino acid sequence of 6× His tag and a cysteine residue added to the carboxyl terminus of human NK1
SEQ ID NO: 6: Nucleotide sequence of human HGF gene
SEQ ID NO: 7: Amino acid sequence of human HGF
SEQ ID NOs: 8 to 16: Amino acid sequence of an ADAM17-sensitive peptide
SEQ ID NOs: 17 to 20: Amino acid sequence of a thrombin-sensitive peptide cm 1-7. (canceled)

8. A polyethylene glycol-modified form of a hepatocyte growth factor or an active fragment thereof, wherein the polyethylene glycol(s) is/are bound to any end of the hepatocyte growth factor or the active fragment thereof via (a) protease-sensitive peptide(s).

9. The polyethylene glycol-modified form according to claim 8, wherein the protease-sensitive peptide(s) is/are (an) ADAM17-sensitive peptide(s) or (a) thrombin-sensitive peptide(s).

10. The polyethylene glycol-modified form according to claim 8, wherein the protease-sensitive peptide(s) has/have an amino acid sequence represented by any one of SEQ ID NOs: 8 to 20 in the sequence listing.

11. The polyethylene glycol-modified form according to claim 8, wherein (a) number-average molecular weight(s) of the polyethylene glycol(s) is/are 20000 to 100000.

12. The polyethylene glycol-modified form according to claim 8, wherein the active fragment of the hepatocyte growth factor is NK1.

13. The polyethylene glycol-modified form according to claim 8, wherein the active fragment of the hepatocyte growth factor has the amino acid sequence represented by SEQ ID NO: 2 in the sequence listing.

14. A medicament comprising the polyethylene glycol-modified form according to claim 8 as an active ingredient.
